(19) United States
(12) Patent Application Publication
(10) Pub. No.: US 2015/0307424 A1
(43) Pub. Date: Oct. 29, 2015

(54) PROCESS FOR THE PRODUCTION AND SEPARATION OF MANNITOL AND SORBITOL FROM A MIXTURE WHICH WAS OBTAINED BY HYDROGENATION OF A PRECURSOR

(71) Applicant: SYRAL BELGIUM NV, Aalst (BE)

(72) Inventors: Frank VAN LANCKER, Aalst (BE); Liesbeth LEHOUCQ, Aalst (BE)

(21) Appl. No.: 14/646,958

(22) PCT Filed: Nov. 20, 2013
(86) PCT No.: PCT/IB2013/002597

§ 371 Date: May 22, 2015

(30) Foreign Application Priority Data
Nov. 22, 2012 (BE) BE-2012/0791

(51) Int. Cl. C07C 29/78; C07C 29/141

(52) U.S. Cl. CPC C07C 29/78; C07C 29/141

(57) ABSTRACT

An improved method for the processing of substrates containing a starting material (precursor) of mannitol, wherein all of the fractions obtained can be recovered, thus eliminating waste.

The present invention concerns an improved method for the processing of substrates containing a starting material (precursor) of mannitol, wherein all of the fractions obtained can be recovered, thus eliminating waste.

Mannitol is a polyol (sugar alcohol) widely used as a natural sweetener, anti-caking agent, and/or filler. It is the hydrogenation product of fructose or mannose. During hydrogenation of fructose, only 50% of the substance is converted to mannitol, while the remainder is hydrogenated into sorbitol. Mannose is not commercially available per se, but can be produced from glucose by means of a chemical isomerisation process. A mixture of glucose and mannose is thus obtained that is then hydrogenated. Suitable and economically available substrates containing a mannitol precursor to be used for the production of mannitol may be selected among the following: sucrose, invert sugar (a mixture of roughly identical amounts of glucose and fructose), HFCS, fructose and glucose and/or polymers rich in fructose or mannose.

Numerous processes for the production of mannitol using the various substrates mentioned above as starting materials have already been described in prior art. In a number of patent publications, a fructose-rich sugar solution (such as invert sugar) is hydrogenated, after which mannitol is crystallised from the reaction mixture and a syrup rich in sorbitol together with considerable amounts of mannitol, and sometimes other impurities, is left as a residue. Examples of such patent publications include AU 3771172, EP 0202168 and U.S. Pat. No. 3,632,656. However, most of the publications do not go into further detail as to what specifically takes place with respect to the so-called sorbitol-rich fraction.

In U.S. Pat. No. 3,864,406, a method is described for separating a mixture of sorbitol and mannitol obtained by hydrogenation of invert sugar. In this method, the mixture is subjected to chromatographic fractionation, yielding highly pure fractions of mannitol and sorbitol. The described method is non-continuous and results in considerable dilution of the various fractions.

A more recent method is described in international patent WO 2012/045985. This publication describes a method that uses invert sugar as the raw material for the production of a sorbitol syrup having a total content of reducing sugar of not more than 0.2% and a mannitol content of less than 1%. In this method, sucrose is hydrolysed in an invert sugar solution that is separated by simulated moving bed (SMB) technology into a glucose syrup having a sugar content of at least 99.3%, and preferably 99.7%, on the one hand and a syrup rich in fructose on the other. The glucose syrup is then hydrogenated into high-purity sorbitol syrup. According to the same application, the fructose syrup can be separately hydrogenated and further processed in order to separate out the mannitol. Another method of using this fructose fraction consists of subjecting it to isomerisation to obtain an invert syrup, which can then be subjected in combination with the starting material to the chromatography step. However, WO 2012/045985 does not concern the simultaneous production of high-purity sorbitol and crystalline mannitol.

It is clear that in cases where the fructose-rich subfraction in WO 2012/045985 is to be used to produce mannitol, a parallel hydrogenation step will be required. This can then be followed by a crystallisation step in which crystalline mannitol and a second sorbitol-rich fraction are obtained. An alternative method can comprise chromatographic separation of the hydrogenated material followed by crystallisation of the mannitol fraction.

It is clear from the above that the production of high-purity sorbitol (<0.2% total reducing sugars) and crystalline mannitol from a source (substrate) containing a precursor (starting material) of mannitol comprises at least four steps, specifically at least one chromatographic separation, two hydrogenations, and a crystallisation step. Within the scope of the present invention, the term “total reducing sugars” refers to the amount of reducing sugars as determined by Bertrand's gravimetric method after acidic treatment of the sorbitol fraction.

German patent publication DE 196 12 826 describes an alternative method for the production of high-purity sorbitol. According to this application, the high-purity sorbitol obtained via this process must contain at least 99.5% sorbitol and less than 0.15% total reducing sugars. In this method, a glucose syrup containing 97 to 99% glucose based on dry matter (DM) is used as a starting material. The described process comprises a chromatographic separation method in which a sorbitol solution containing 98-99% sorbitol is processed in a specified chromatographic column configuration in which three fractions are obtained. Conversion from one fraction to the next is performed depending on the refractive index of the eluates.

The method described in DE 196 12 826 is disadvantageous because an extremely large amount of water is required to carry out the process, while the glucose syrup used as a substrate must also possess an initially high purity. In addition, the processing capacity of the system is quite low.

The high-purity sorbitol obtained in DE 196 12 826 and WO 2012/045985 is particularly well-suited for use in chemical reactions requiring high thermal stability (such as the production of isosorbide, sorbitan esters, or polyether polyols).

In view of the above, it is clear that the object of the present invention can be defined as a technically feasible method for the simultaneous production of a high-purity sorbitol fraction and crystalline mannitol together with a second sorbitol-rich fraction using a substrate containing a precursor of mannitol. The method should comprise only a limited number of steps, it should be conducted using only simple, commonly-known production equipment, it should be applicable to various substrates, and all of the fractions obtained by the method should be recovered as high-added-value products.

The object of the invention is achieved by providing a method for the separation of a substrate containing a mannitol precursor into several fractions comprising the following steps:
(a) hydrogenation of the aforementioned substrate to a hydrogenated substrate;
(b) chromatographic separation of the hydrogenated substrate by a continuous process into:
   a mannitol-rich fraction,
   a first sorbitol fraction of high purity containing less than 0.2% total reducing sugars, and preferably less than 0.15% total reducing sugars, and
   a second sorbitol fraction;
(c) crystallisation of the mannitol-rich fraction into a third fraction of crystalline mannitol, in which a mother liquor is formed;
(d) re-addition of this mother liquor to the hydrogenated substrate.

Step (b), specifically chromatographic separation of the hydrogenated substrate, takes place by means of a continuous process, i.e., chromatographic separation takes place by conducting repeated cycles of steps without interruption.

Within the scope of the present invention, the term “mannitol-rich” is to be understood as referring to a fraction containing at least 60% mannitol based on dry matter, preferably at least 64% mannitol DM, and more preferably at least 67% mannitol DM.

The first sorbitol fraction (X) preferably contains at least 98% sorbitol. In particular, the first sorbitol fraction should contain <0.2%, and more preferably <0.15% total reducing sugars.

According to a more preferred embodiment of the method according to the invention, at least two and at most six resin beds connected in series are used for chromatographic separation. Specifically, one of the following separation protocols is used for chromatographic separation: SSMB, ISMB, MCI or NMCI. The choice of the specific protocol depends on the substrate.

The substrate used for this method is preferably a carbohydrate composition containing fructose and/or mannose. Specifically, the substrate is selected among one of the following compositions: invert sugar, glucose-fructose syrup, fructose-enriched syrups, inulin hydrolysate, or mannose-containing syrup. Mannose-containing syrup can be obtained by chemical isomerisation of glucose or by hydrolysis of glucomannans. After hydrogenation of these substrates, refining may be conducted prior to the chromatographic step (step b) in order to increase the stability of the chromatographic separation process.

Typical resins used for chromatographic separation of polyols are sulphonated polystyrenes in calcium form, with a suitable degree of divinylbenzene (DVB) crosslinking that imparts physical stability to the resin. The sulphonic acid function of the resin particles causes swelling in aqueous media. The resulting microporous resin beads can absorb water and non-ionically dissolved substances. Typical ranges of crosslinking are 2-15%, with crosslinking of 3-8% being preferred. Known examples of such resins are DIAION UBK 555 (Mitsubishi Chemical Corporation) and DOWEX*MONOSPHERE*N279 Ca (The Dow Chemical Company).

According to a particular embodiment of the method of the invention, hydrogenated compositions subjected to chromatographic separation contain, on a dry matter basis, approximately 20-60% mannitol, approximately 40-80% sorbitol, and 0.1-5% other sugar alcohols, with the total of the various components not exceeding 100%. More preferably, hydrogenated compositions subjected to chromatographic separation contain, on a dry matter basis, 23-30% mannitol, 70-78% sorbitol and 0.1-5% other sugar alcohols.

In an alternative embodiment of the method according to the present invention, steps (c) and (d) are omitted, and following step (a), the hydrogenated substrate is subjected to a crystallisation step in which a third fraction is formed with crystalline mannitol, and a mother liquor. Subsequently the mother liquor is subjected to chromatographic separation according to step (b), after which the mannitol-rich subfraction formed is recirculated to the hydrogenated substrate for crystallisation.

The invention will now be explained in further detail based on the following description and detailed examples of the method according to the present invention. As the purpose of this description is simply to provide explanatory examples, it is by no means to be interpreted as limiting the scope of application of the invention or the patent rights specified in the claims.

In this description, the attached figures will be referred to by means of reference numbers, with the figures being as follows:

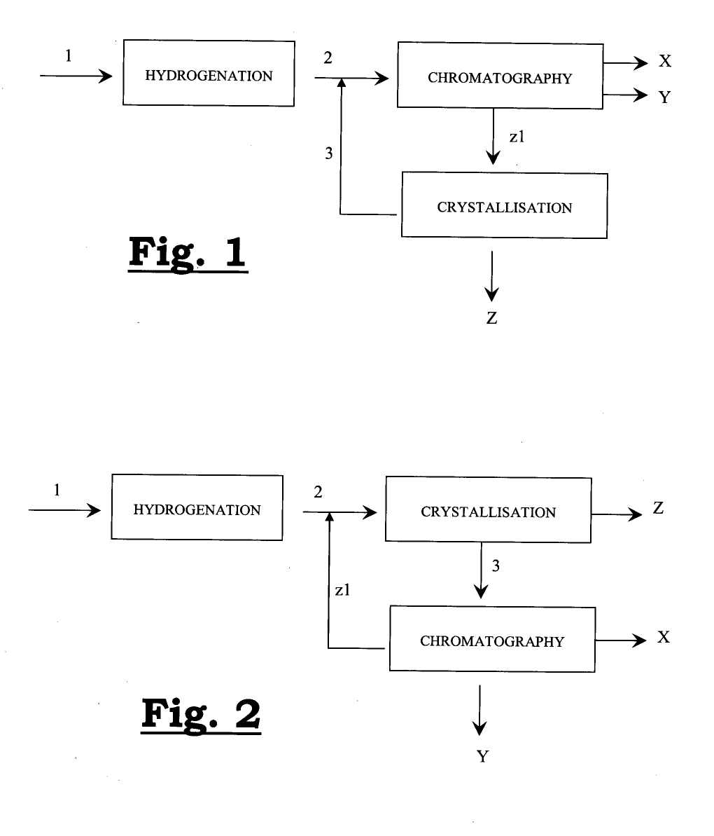

FIG. 1 is a schematic diagram of the method according to the invention;

FIG. 2 is a schematic diagram of the alternative embodiment of the method according to the invention;

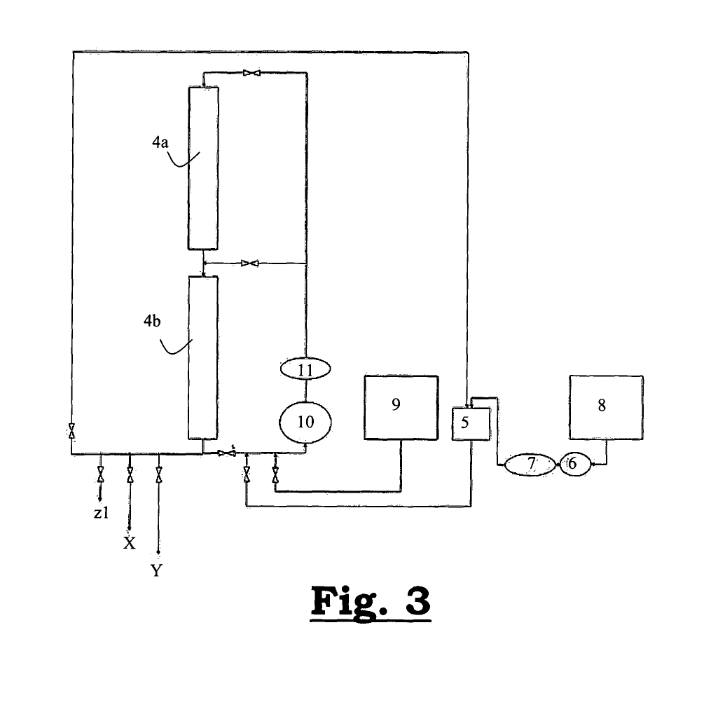

FIG. 3 is a schematic diagram of a device suitable for carrying out chromatographic separation according to the method of the invention.

The method according to the present invention is suitable for processing a substrate (1) containing a mannitol precursor, such as invert sugar, glucose-fructose syrup, fructose-enriched syrups, hydrolysates of inulin, and mannose-containing syrup in such a manner that a high-purity sorbitol fraction (X), crystalline mannitol (Z), and a sorbitol-rich fraction (Y) can be produced simultaneously.

According to the method of the invention, the substrate (1) is first hydrogenated to obtain a hydrogenated substrate (2). After hydrogenation, this hydrogenated substrate (2) is subjected to a chromatographic separation step that yields a mannitol-rich fraction (z1), a high-purity sorbitol fraction (X) containing a minimum of 98% sorbitol and <0.2%, and preferably <0.15% total reducing sugars, and a second sorbitol-rich fraction (Y). The mannitol-rich fraction (z1) is then subjected to a crystallisation step. After this crystallisation step, the crystalline mannitol (Z) is separated from the mother liquor (3), which in turn is added to the hydrogenated substrate (2). This method is shown schematically in FIG. 1.

According to another embodiment of the method of the invention, the hydrogenated substrate (2) is first subjected to crystallisation, with a portion of the mannitol present being recovered in crystalline form, and the mother liquor is then subjected to chromatographic separation. This mother liquor (3) is thus separated into a high-purity sorbitol fraction (X) containing at least 98% sorbitol and <0.2%, and preferably <0.15% total reducing sugars, a second sorbitol-rich fraction (Y), and a mannitol-enriched fraction (z1) that is recirculated to the hydrogenated substrate for crystallisation. This is schematically illustrated in FIG. 2.

Chromatographic separation is conducted using equipment having a limited number of resin beds connected in series, with the number of beds varying from 2 to 6. Various separation protocols may be used in this process, such as SSMB, ISMB, MCI or NMCI, depending on which protocol is most suitable for the substrate used.

According to a particular embodiment of the method of the invention, the chromatographic separation process involves the use of two resin beds connected in series (4a and 4b), with the method comprising repetition of the following steps (cf. FIG. 3):
Introduction of the syrup to be separated at the top of the first resin bed (4a) and simultaneous extraction via the outlet port of the second resin bed (4b) of a mannitol-rich fraction (z1) of the same volume. The aforementioned syrup is already hydrogenated and is supplied from a so-called syrup feeding tank (5). This feeding tank (5) is connected via a duct equipped with a feeding pump (6) and flow meter (7) to a storage tank (8) for this syrup.
Introduction of demineralised water at the top of the second resin bed (4b) while circulating the same volume collected at the outlet port of the second resin bed (4b) to the feeding tank (5) of the chromatographic separation system. The system is equipped with a demineralised water storage tank (9) in order to ensure a sufficient supply of demineralised water.
Connection of the discharge port of the second resin bed (4b) to the inlet port of the first resin bed (4a) and circulation of the product via a circulation pump (10) equipped with a flow meter (11),
Introduction of demineralised water at the top of the first resin bed (4a) and simultaneous collection at the outlet port of the second resin bed (4b) of a high-purity sorbitol fraction (X) of the same volume,
Introduction of demineralised water at the top of the first resin bed (4a) and simultaneous collection at the outlet port of the second resin bed (4b) of a second sorbitol-rich fraction (Y) of the same volume,
Connection of the discharge port of the second resin bed (4b) to the inlet port of the first resin bed (4a) and circulation of the product via the circulation pump (10).

A number of examples are given in the following in order to further clarify the method according to the invention:

### EXAMPLE 1

### Production of Mannose-Rich Syrup

1 litre of Dowex MSA-1 (a macroporous, strongly basic anion exchange resin manufactured by the Dow Chemical Company) is immersed in an aqueous solution of 250 g/L of sodium molybdate for 12 h and gently stirred. During this period, a sufficient amount of a 1N solution of hydrochloric acid is added to maintain a pH of 3.5. The resin is then placed in a double-walled column and washed with demineralised water until all of the molybdate remaining in the solution is removed and then readjusted to a pH of 3.5.

The column is now heated to 85° C. and fed with 40 Bx glucose syrup (96% dextrose DM) at 0.5 BV/hr. The effluent of the column is collected and simultaneously analysed for mannose content. The feeding rate of the molybdate-packed column is adjusted so as to maintain a mannose content of 26%±1% DM at the outlet of the column.

### EXAMPLE 2

Production of syrup rich in sorbitol and mannitol from chemically isomerised high-dextrose syrup:

The syrup from Example 1 is purified by passage over a Dowex 88 resin bed in H+ form (a strongly acidic cation exchange resin manufactured by the Dow Chemical Company) and a Dowex 66 resin bed in free amine form (a weakly basic anion exchanger manufactured by the Dow Chemical Company) connected in series.

The refined syrup is concentrated to 50 Bx, and 3% w/w of 5% Ru/CP catalyst 9017 (BASF) is added. The suspension is added to a reactor and heated to 105° C. while stirring at a pressure of 40 bar H₂ until consumption of H₂ stops. The reactor is cooled down, the catalyst is filtered off, and the clear syrup is introduced at 0.5 BV/hr over a Dowex MSA-1 resin bed (a macroporous, strongly basic anion exchange resin manufactured by the Dow Chemical Company) in OH— form at 60° C. in order to remove the remaining reducing sugars. The syrup is further refined by being passed at 30° C. and 2 BV/hr over a Lewatit S8528 resin bed in H+ form (a weakly acidic cation exchange resin manufactured by Lanxess). The pH of the refined syrup is adjusted to 6.0 by adding 0.05 N NaOH.

### EXAMPLE 3

### Production of Syrup Rich in Sorbitol and Mannitol from High-Fructose Syrup

Isosweet 111, a high-fructose syrup produced by Syral Belgium N V, is diluted to 50 Bx and 3% w/w of 5% Ru/CP 9017 catalyst (BASF) is added. The suspension is added to a reactor and heated to 105° C. while stirring at an H₂ pressure of 40 bar until consumption of H₂ stops. The reactor is cooled down, the catalyst is filtered off, and the clear syrup is introduced at 0.5 BV/hr over a Dowex MSA-1 resin bed (a macroporous, strongly basic anion exchange resin manufactured by the Dow Chemical Company) in OH— form at 60° C. in order to remove the remaining reducing sugars. The syrup is further refined by feeding at 30° C. and 2 BV/hr over a Lewatit S8528 resin bed in H+ form (a weakly acidic cation exchange resin manufactured by Lanxess). The pH of the refined syrup is adjusted to 6.0 by adding 0.05 N NaOH.

### EXAMPLE 4

### Simultaneous Production of High-Purity Sorbitol, Non-Crystallising Sorbitol, and Crystalline Mannitol from Mannitol Syrup Obtained by Chemical Isomerisation of Glucose

A syrup produced according to Example 2 and containing 26.3% mannitol and 65.8% sorbitol, with the remainder being composed of DP2 and higher polyols and cracking products, is concentrated to 55 Bx and separated into 3 fractions by chromatographic separation.

The chromatographic separation equipment is shown schematically in FIG. 3 and comprises 2 double-walled columns (4a and 4b) in series, each 1 m in height, with a usable internal volume of 2.35 L, which are stored at 80° C. The columns are filled with DIAION UBK 555 (Mitsubishi Chemical Corporation) separation resin. The system further comprises 3 tanks temperature-controlled to 80° C.: a 20 L syrup storage tank (8), a 2 L syrup feeding tank (5), and a 50 L demineralised water tank (9). The system further comprises a syrup metering pump (6), a circulation pump (10), a flow meter (7, 11), and the required electromagnetic valves and connecting ducts.

The separation process comprises repetition of the following steps:

1. 0.5 L of mannitol syrup from the syrup feeding tank is introduced at 1.4 L/hr at the top of column 1 (4a), while at the same time, 0.5 L of product fraction (z1) (mannitol fraction) is collected at the outlet port of column 2 (4b).
2. 0.126 L of demineralised water from the demineralised water tank (9) is introduced at 1.4 L/hr at the top of column 2 (4b), and at the same time, 0.126 L of product from the outlet port of column 2 (4b) is recirculated to the syrup feeding tank (5) of the chromatographic separation system.
3. The inlet port of column 1 and outlet port of column 2 are connected via the circulation pump, and the product is circulated via the outlet port of column 2 to the inlet port of column 1 for 26 minutes at a flow rate of 1.4 L/hr.
4. 0.423 L of demineralised water is added at the top of column 1 at 1.4 L/hr, and at the same time, 0.423 L of product fraction 2 (high-purity sorbitol) is collected at the outlet port of column 2.
5. 0.987 L of demineralised water is added at the top of column 1 at 1.4 L/hr, and at the same time, 0.987 L of product fraction 3 (non-crystallising sorbitol) is collected at the outlet port of column 2 (4b).
6. The inlet port of column 1 and the discharge port of column 2 are connected via the circulation pump, and the product is circulated via the outlet port of column 2 to the inlet port of column 1 for 53 minutes at a flow rate of 1.4 L/hr.

After conducting 5 cycles of the above 6 steps, all of the fractions are stabilised with respect to Bx and composition. During the following 32 cycles, all of the mannitol fractions (z1 fractions) are collected and concentrated in mannitol syrup having a content of 57% DM and purity of 67.9%.

This syrup is placed in a 3 L lab crystalliser (brand name Labo Service Belgium) and heated to 80° C. for three h. After this, the crystalliser is cooled while gently stirring at a rate of 1° C./hr. D-mannitol crystals (Merck 1.05980.9050) are added as a seed material when the temperature in the crystalliser reaches 72° C. The content of seed material is 0.2% DM of the feed solution. Cooling is continued at 1° C./hr to 30° C. The crystallised mass is transferred to a Rousselet-Robatel RC30VxR centrifuge, and the crystals and mother liquor are separated. The crystal cake is washed three times at room temperature with 30 mL demineralised water per kg of crystals.

The mother liquor and washing solution are collected and mixed at a ratio of 1/3.8 parts DM/DM with the mannitol syrup produced as in Example 2. The resulting syrup has a mannitol content of 29.3% and is concentrated to 55 Bx.

This syrup is now used as feed solution for another process of 6-step chromatographic separation as described above. The mannitol fractions (no. 1 fractions) from the first 5 cycles are removed. The mannitol fractions of the following 25 cycles are collected and concentrated into a mannitol syrup with 71% purity and a content of 56% DM at a similar mannitol to water ratio as that of the mannitol syrup having a content of 57% DM and 67.9% purity. The new mannitol syrup is now subjected to the same cycle of crystallisation, crystal separation, and washing as described above. The mother liquor and washing solution are collected and mixed at a ratio of 1/3.8 parts DM/DM with the mannitol syrup produced in Example 2. The resulting syrup has a mannitol purity of 30.0% DM and is concentrated to 55 Bx.

This process, which comprises:

a) production via chromatography of a mannitol fraction suitable for crystallisation,
b) production during crystallisation, crystal separation, and washing of a combined mother liquor and washing solution fraction,
c) mixing of this combined mother liquor and washing solution fraction at a ratio of 1/3.8 parts DM/DM with a mannitol syrup produced as in Example 2, and
d) concentration of the obtained mixture to 55 Bx prior to chromatographic separation under a) above, is repeated two more times until all of the fractions show a stable composition. After completion of these repetitions, the following yields and purities are found:
mannitol crystals: 26% yield (crystals DM/feed solution DM) and 98.8% purity,
high-purity sorbitol: 40% yield (sorbitol DM/feed solution DM), 98.6% purity, and 0.2% total reducing sugars,
non-crystallising sorbitol: 34% yield (sorbitol DM/feed solution DM) and 77.8% purity.

### EXAMPLE 5

Simultaneous production of high-purity sorbitol, non-crystallising sorbitol, and crystalline mannitol from hydrogenated HFCS syrup.

A syrup produced according to Example 3 and containing 21.3% mannitol and 73.8% sorbitol DM, with the remainder composed of DP2 and higher polyols and cracking products, is concentrated to 55 Bx and separated into 3 fractions by chromatographic separation. The chromatographic separation equipment is the same as that described in Example 4.

The separation process comprises repetition of the following steps:

1. 0.45 L of mannitol syrup from the syrup feeding tank is introduced at a flow rate of 1.4 L/hr at the top of column 1, while at the same time, 0.45 L of product fraction (z1) (mannitol fraction) is collected at the outlet port of column 2 (4b).
2. 0.176 L of demineralised water from the demineralised water tank is introduced at 1.4 L/hr at the top of column 2, and at the same time, 0.176 L of product from the outlet port of column 2 is recirculated to the syrup feeding tank (5) of the chromatographic separation system.
3. The inlet port of column 1 and the discharge port of column 2 are connected via the circulation pump, and the product is circulated via outlet column 2 to the inlet port of column 1 for 26 minutes at a flow rate of 1.4 L/hr.
4. 0.380 L of demineralised water is added at the top of column 1 at 1.4 L/hr, and at the same time, 0.380 L of product fraction (X) (high-purity sorbitol) is collected at the outlet port of column 2 (4b).
5. 1.030 L of demineralised water is added at the top of column 1 at 1.4 L/hr, and at the same time, 1.030 L of product fraction (Y) (non-crystallised sorbitol) is collected at the outlet port of column 2 (4b).
6. The inlet port of column 1 (4a) and the discharge port of column 2 (4b) are connected via a circulation pump, and the product is recirculated via outlet column 2 (4b) to the inlet port of column 1 (4a) for 53 minutes at 1.4 L/h.

After conducting 5 cycles of the preceding 6 steps, all of the fractions are stabilised with respect to Bx and composition. During the following 32 cycles, all of the mannitol fractions (z1 fractions) are collected, and a concentrated mannitol syrup with a content of 59.3% DM and purity of 61.3% is obtained.

This syrup is placed in a 3 L lab crystalliser (brand name Labo Service Belgium) and heated to 80° C. for three h. After this period, the crystalliser is cooled while gently stirring at a rate of 1° C./hr. D-mannitol crystals (Merck 1.05980.9050) are added as a seed material when the temperature in the reaches 72° C. The content of seed material is 0.2% DM of the feed solution. Cooling is continued at 1° C./hr to 30° C. The crystallised mass is transferred to a Rousselet-Robatel RC30VxR centrifuge, and the crystals and mother liquor are separated. The crystal cake is washed three times at room temperature with 30 mL demineralised water per kg of crystals. The mother liquor and washing solution are collected and mixed at a ratio of 1/4 parts DM/DM with the mannitol syrup produced as in Example 3. The resulting syrup has a mannitol content of 23.9% and is concentrated to 55 Bx.

This syrup is now used as feed solution for next 6-step chromatographic separation as described above. The mannitol fractions (z1 fractions) of the first 5 cycles are removed. The mannitol fractions of the following 25 cycles are collected and concentrated to a mannitol syrup having a purity of 64.7% and content of 58% DM, with a similar ratio of mannitol to water as that of the mannitol syrup having a content of 59.3% DM and 61.3% purity. The new mannitol syrup is now subjected to the same cycle of crystallisation, crystal separation, and washing as described above. The mother liquor and washing solution are collected and mixed at a ratio of 1/4 parts DM with the mannitol syrup produced in Example 2. The resulting syrup, with a mannitol content of 24.5%, is concentrated to 55 Bx.

This process, which comprises the following:

e) production by chromatography of a mannitol fraction suitable for crystallisation, production during crystallisation, crystal separation, and washing of a combined mother liquor and washing solution fraction,
g) mixing of this combined mother liquor and washing solution fraction at a ratio of 1/4 parts DM with a mannitol syrup produced as in Example 3, and
h) concentration of the mixture obtained to 55 Bx before it is subjected to the chromatographic separation of step e) above, is repeated two more times until all of the fractions show a stable composition.

The following yields and purities are found on completion of these repetitions:
mannitol crystals: 21% yield (crystals DM/feed solution DM) and 98.8% purity;
high-purity sorbitol: 43% yield (sorbitol DM/feed solution DM), 98.8% purity, and 0.15% total reducing sugars;
non-crystallising sorbitol: 36% yield (sorbitol DM/feed solution DM) and 86.3% purity.

1. Method for the separation of a substrate containing fructose and/or mannose that contains a mannitol precursor into several fractions, comprising the following steps:
a) hydrogenation of said substrate into a hydrogenated substrate;
b) chromatographic separation of the hydrogenated substrate by a continuous process into:
a mannitol-rich fraction,
a first sorbitol fraction having high purity and containing less than 0.2% total reducing sugars,
a second sorbitol fraction;
c) crystallisation of the mannitol-rich fraction into a third fraction of crystalline mannitol, with a mother liquor being formed; and
d) readdition of the mother liquor to the hydrogenated substrate.

2. Method according to claim 1, characterised in that the mannitol-rich fraction contains at least 60% mannitol DM.

3. Method according to claim 1, characterised in that the first sorbitol fraction contains at least 98% sorbitol.

4. Method according to claim 1, characterised in that at least two and at most six resin beds connected in series are used for chromatographic separation.

5. Method according to claim 1, characterised in that one of the following separation protocols is used for chromatographic separation: SSMB, ISMB, MCI or NMCI.

6. Method according to claim 1, characterised in that the aforementioned substrate is a carbohydrate composition containing fructose and/or mannose.

7. Method according to claim 1, characterised in that the aforementioned substrate is selected among one of the following compositions: invert sugar, glucose-fructose syrup, fructose-enriched syrups, inulin hydrolysates, or mannose-containing syrup.

8. Method according to claim 1, characterised in that the hydrogenated substrate contains, on a dry weight basis, 20-60% mannitol, 40-80% sorbitol, and 0.1-5% other sugar alcohols, with the total of the various components not exceeding 100%.

9. Method according to claim 1, characterised in that the hydrogenated substrate contains, on a dry weight basis, 23-30% mannitol, 70-78% sorbitol, and 0.1-5% other sugar alcohols, with the total of the various components not exceeding 100%.

10. Method for the separation of a substrate containing fructose and/or mannose that contains a mannitol precursor into several fractions, comprising the following steps:
a) hydrogenation of the aforementioned substrate into a hydrogenated substrate;
b) crystallisation of the hydrogenated substrate, thus forming a third fraction containing crystalline mannitol and a mother liquor;
c) chromatographic separation of the mother liquor formed into:
a mannitol-rich fraction,
a first sorbitol fraction having high purity and containing less than 0.2% total reducing sugars, and
a second sorbitol fraction; and
d) readdition of the mannitol-rich subfraction formed to the hydrogenated substrate for crystallisation.
